(19) United States
(12) Patent Application Publication
(10) Pub. No.: US 2012/0253199 A1
(43) Pub. Date: Oct. 4, 2012

(54) ULTRASONIC PROBE AND ULTRASONIC PROBE MANUFACTURING METHOD

(75) Inventor: Minoru AOKI, Nasushiobara-shi (JP)

(73) Assignees: TOSHIBA MEDICAL SYSTEMS CORPORATION, OTAWARA-SHI (JP); KABUSHIKI KAISHA TOSHIBA, TOKYO (JP)

(21) Appl. No.: 13/427,709

(22) Filed: Mar. 22, 2012

(30) Foreign Application Priority Data
Mar. 29, 2011 (JP) 2011-073244

(51) Int. Cl. A61B 8/00; H04R 31/00

(52) U.S. Cl. 600/459; 29/594

(57) ABSTRACT

According to one embodiment, an ultrasonic probe includes a transducer element, a backing material, and a buffer layer. The transducer element vibrates to transmit and receive an ultrasonic wave. The buffer layer is provided on the back side of the transducer element. The backing material is provided on the back side of the buffer layer and damps an ultrasonic wave from the transducer element. The buffer layer has a Poisson ratio larger than that of the backing material.

### CROSS-REFERENCE TO RELATED APPLICATIONS

This application is based upon and claims the benefit of priority from prior Japanese Patent Application No. 2011-073244, filed Mar. 29, 2011, the entire contents of which are incorporated herein by reference.

### FIELD

Embodiments described herein relate generally to an ultrasonic probe and an ultrasonic probe manufacturing method.

### BACKGROUND

An ultrasonic probe has a transducer element to transmit and receive an ultrasonic wave. An acoustic matching layer to reduce an acoustic impedance mismatch between the piezoelectric transducer element and a living body is provided on a front side (surface on the side of a living body contact surface of the ultrasonic probe) of the transducer element and a backing material to damp an ultrasonic wave from the transducer element is provided on the back side thereof. When transmitting or receiving an ultrasonic wave, the transducer element vibrates mechanically. This vibration also causes the backing material to vibrate mechanically. Mechanical vibrations of the backing material produce noise in acoustic properties of the ultrasonic probe, causing degradation of acoustic properties.

An object of the present embodiment is to provide an ultrasonic probe that prevents degradation of acoustic properties involved in vibration of a transducer element and an ultrasonic probe manufacturing method.

### BRIEF DESCRIPTION OF THE DRAWINGS

FIG. 1 is a diagram showing a schematic structure of an ultrasonic probe according to the present embodiment;

FIG. 2A is a diagram showing an acoustic simulation result of an ultrasonic probe in a conventional structure;

FIG. 2B is a diagram showing an ultrasonic probe in the conventional structure of FIG. 2A;

FIG. 3A is a diagram showing an acoustic simulation result of the ultrasonic probe according to the present embodiment;

FIG. 3B is a diagram showing an ultrasonic probe according to the present embodiment of FIG. 3A;

FIG. 4 is a diagram showing a typical flow of a manufacturing process of an ultrasonic probe according to Example 1 of the present embodiment;

FIG. 5 is a diagram illustrating step SA1 in FIG. 4;

FIG. 6 is a diagram illustrating step SA2 in FIG. 4;

FIG. 7 is a diagram illustrating step SA3 in FIG. 4;

FIG. 8 is a diagram illustrating step SA4 in FIG. 4;

FIG. 9 is a diagram illustrating step SA5 in FIG. 4;

FIG. 10 is a diagram showing the typical flow of the manufacturing process of an ultrasonic probe according to Example 2 of the present embodiment;

FIG. 11 is a diagram illustrating step SB1 in FIG. 10;

FIG. 12 is a diagram illustrating step SB2 in FIG. 10;

FIG. 13 is a diagram illustrating step SB3 in FIG. 10;

FIG. 14 is a diagram illustrating step SB4 in FIG. 10;

FIG. 15 is a diagram illustrating step SB5 in FIG. 10;

FIG. 16 is a diagram illustrating step SB6 in FIG. 10;

FIG. 17 is a diagram showing the schematic structure of an ultrasonic probe according to Modification 1; and

FIG. 18 is a diagram showing the schematic structure of an ultrasonic probe according to Modification 2.

### DETAILED DESCRIPTION

In general, according to one embodiment, an ultrasonic probe includes a transducer element, a backing material, and a buffer layer. The transducer element vibrates to transmit and receive an ultrasonic wave. The buffer layer is provided on the back side of the transducer element. The backing material is provided on the back side of the buffer layer and damps an ultrasonic wave from the transducer element. The buffer layer has a Poisson ratio larger than that of the backing material.

The ultrasonic probes and the manufacturing methods of ultrasonic probes according to the present embodiment will be described below with reference to the drawings.

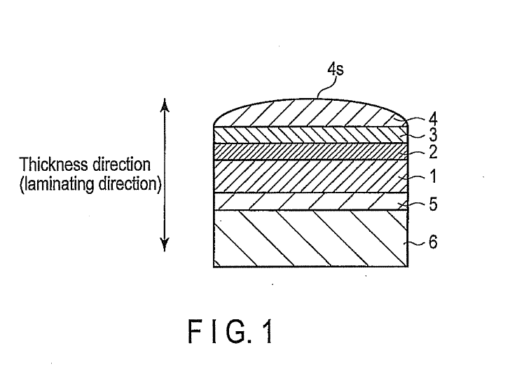

FIG. 1 is a diagram showing a schematic structure of an ultrasonic probe according to the present embodiment. As shown in FIG. 1, the ultrasonic probe includes a transducer unit 1 that transmits and receives an ultrasonic wave. A first acoustic matching layer 2 is provided on a front side (surface on the side of a living body contact surface 4s) of the transducer unit 1. A second acoustic matching layer 3 is provided on the front side of the first acoustic matching layer 2. An acoustic lens 4 is provided on the front side of the second acoustic matching layer. A buffer layer 5 is provided on a back side (surface on the opposite side of the living body contact surface 4s) of the transducer unit 1. A backing material 6 is provided on the back side of the buffer layer 5. The laminating direction of the backing material 6, the buffer layer 5, the transducer unit 1, the first acoustic matching layer 2, the second acoustic matching layer 3, and the acoustic lens 4 is defined as a thickness direction.

The transducer unit 1 has a plurality of transducer elements (not shown in FIG. 1) arranged one-dimensionally or two-dimensionally. Each transducer element includes a piezoelectric element (not shown in FIG. 1) formed of a piezoelectric material, an electrode formed on the front side of the piezoelectric element (hereinafter, referred to as a front-side electrode), and an electrode formed on the back side of the piezoelectric element (hereinafter, referred to as a back-side electrode). The piezoelectric material is, for example, piezoelectric ceramic whose acoustic impedance is 30 Mrayl (Mrayl=10⁶ kg/m²s) or more. The transducer element vibrates after receiving the supply of a driving signal from the main body of ultrasonic diagnostic equipment to transmit an ultrasonic wave. The generated ultrasonic wave is reflected by a subject. The reflected ultrasonic wave is received by the transducer element. When an ultrasonic wave is received, the transducer element vibrates to generate an electric signal. The generated electric signal is transmitted to the main body of ultrasonic diagnostic equipment. The transducer element vibrates in the thickness direction. In other words, the vibration mode of the transducer element is a thickness vibration mode.

The first acoustic matching layer 2 and the second acoustic matching layer 3 are provided to reduce an acoustic impedance mismatch between the transducer unit 1 and a living body. The first acoustic matching layer 2 and the second acoustic matching layer 3 are each formed so as to have acoustic impedances between the acoustic impedance of the transducer unit 1 and the acoustic impedance of the living body. The first acoustic matching layer 2 is formed so as to have a higher acoustic impedance than the second acoustic matching layer 3. The acoustic impedance of a living body is generally 1.5 Mrayl. The first acoustic matching layer 2 has a plurality of first acoustic matching elements (not shown in FIG. 1) arranged one-dimensionally or two-dimensionally. Similarly, the second acoustic matching layer 3 has a plurality of second acoustic matching elements (not shown in FIG. 1) arranged one-dimensionally or two-dimensionally. Incidentally, acoustic matching layers contained in an ultrasonic probe according to the present embodiment is not limited to the two layers of the first acoustic matching layer 2 and the second acoustic matching layer 3. An ultrasonic probe according to the present embodiment may have one acoustic matching layer or three or more acoustic matching layers.

The acoustic lens 4 is provided to converge ultrasonic waves radiated toward a subject from the transducer unit 1. The acoustic lens 4 is formed so as to have acoustic impedance between the acoustic impedance of the second acoustic matching layer 3 and the acoustic impedance of a living body. The acoustic lens 4 has the living body contact surface 4s brought into contact with a subject.

The buffer layer 5 is provided to damp mechanical vibrations of the transducer unit 1 involved in transmission and reception of an ultrasonic wave. As the material of the buffer layer 5, for example, a layer structure using a polymeric material of polyurethane or polyethylene or a silicon resin material as a base material is used. The buffer layer 5 will be described in detail later.

The backing material 6 is provided to damp acoustic vibration by the transducer unit 1. In other words, the backing material 6 damps an ultrasonic wave radiated in the direction of the backing material 6 from the transducer unit 1. The backing material 6 functions also as a structural holding material of the transducer unit 1. More specifically, if the ultrasonic probe is a linear probe, the backing material 6 is provided to hold a plurality of transducer elements linearly and if the ultrasonic probe is a convex probe, the backing material 6 is provided to hold a plurality of transducer elements in a circular shape of a fixed curvature. As the material of the backing material 6, a rubber material such as polybutadiene and chloroprene is used. The backing material 6 is formed so as to have acoustic impedance that does not cause unnecessary resonance by acoustic matching. Typically, the backing material 6 is formed so as to have an acoustic impedance of 2 to 7 Mrayl.

Next, the buffer layer 5 will be described in detail.

If there is no buffer layer like in a conventional structure, mechanical vibrations of the transducer element are propagated to the backing material, causing the backing material to mechanically vibrate. Mechanical vibration of the backing material causes degradation of acoustic properties of an ultrasonic probe. Also if there is no buffer layer like in a conventional structure, mechanical vibrations of some transducer element are propagated to adjacent piezoelectric transducer elements via the backing material having stiffness. Also due to mechanical vibrations of adjacent piezoelectric transducer elements, an acoustic field of ultrasonic waves radiated from an ultrasonic probe is disturbed, leading to degradation of acoustic properties of the ultrasonic probe.

The buffer layer 5 is provided to prevent mechanical vibrations of a transducer element from being propagated to the backing material 6 or adjacent transducer elements. Thus, the buffer layer 5 is formed so as to have more flexibility than the backing material 6, that is, a Poisson ratio larger than that of the backing material 6. The Poisson ratio is defined as β/α if contracted (or expanded) by β in the horizontal direction when expanded (or contracted) by α per unit length in the thickness direction.

The backing material 6 is provided, as described above, to hold a geometrical arrangement of the transducer unit 1, the first acoustic matching layer 2, and the second acoustic matching layer 3 to prevent a disturbance of the acoustic field of ultrasonic waves. The backing material 6 needs to have an ultrasonic attenuation capacity and an acoustic impedance to prevent unnecessary resonance. The backing material 6 is formed of a material based on a rubber material, but various additives are mixed to meet the ultrasonic attenuation capacity and the constraint of the acoustic impedance. As a result, the Poisson ratio of the backing material 6 is relatively small, that is, the backing material 6 has high stiffness.

To improve the damping effect, the higher the Poisson ratio of the buffer layer 5, the better it is. If the fact that the backing material 6 is formed of a resin material or rubber material and the buffer layer 5 is formed of silicon, urethane, or other resin materials as a base material is taken into consideration, the Poisson ratio of the buffer layer 5 may be set to, for example, 0.4 or more. However, the Poisson ratio of the buffer layer 5 according to the present embodiment is not limited to 0.4 or more. If the damping effect by the buffer layer 5 is obtained, the Poisson ratio of the buffer layer 5 may be less than 0.4.

As described above, the backing material 6 is provided to damp ultrasonic waves radiated in the direction of the backing material 6 from the transducer unit 1. An ultrasonic wave has a physical property of being reflected on a discontinuity surface of the acoustic impedance. In the present embodiment, the buffer layer 5 is provided between the transducer unit 1 and the backing material 6. If the buffer layer 5 and the backing material 6 have different acoustic impedances, an ultrasonic wave is reflected by a boundary surface between the buffer layer 5 and the backing material 6. The acoustic field of ultrasonic waves radiated from an ultrasonic probe according to the present embodiment is disturbed by an ultrasonic wave reflected by the buffer layer 5, leading to degradation of acoustic properties of the ultrasonic probe according to the present embodiment.

To prevent the reflection of an ultrasonic wave by a boundary surface between the buffer layer 5 and the backing material 6 to allow efficient propagation of the ultrasonic wave from the transducer unit 1 to the backing material 6, the acoustic impedance of the buffer layer 5 is designed to substantially match the acoustic impedance of the backing material 6. More specifically, the difference between the acoustic impedance of the buffer layer 5 and the acoustic impedance of the backing material 6 is adjusted to −20% or more and +20% or less. In reality, acoustic impedances of the buffer layer 5 and the backing material 6 are substantially matched by adjusting the acoustic impedance of the buffer layer 5 closer to the acoustic impedance of the backing material 6. For the adjustment method of the acoustic impedance, for example, a microstructure of particles using metallic oxide or ceramic material as a material or the like is mixed with the base material of the buffer layer 5. Incidentally, the difference between the acoustic impedances of the buffer layer 5 and the backing material 6 is not limited to −20% or more and +20% or less. If the frequency of reflection of an ultrasonic wave by a boundary surface between the buffer layer 5 and the backing material 6 can be reduced to some extent, the difference between the acoustic impedances of the buffer layer 5 and the backing material 6 may be −20% or less or +20% or more.

If only improvement of the damping effect of the buffer layer 5 is intended, the thicker the buffer layer 5 is, the better it is. However, if the buffer layer 5 is unnecessarily thick, mechanical stiffness to hold piezoelectric transducer elements declines, making the geometrical arrangement of the piezoelectric transducer elements unstable. As a result, the acoustic field of ultrasonic waves radiated from an ultrasonic probe is disturbed, leading to degradation of acoustic properties of the ultrasonic probe.

Conversely, if the buffer layer 5 is unnecessarily thin, mechanical vibrations of the transducer element cannot be suppressed. As a result, mechanical vibrations are propagated to the backing material 6 and adjacent transducer elements, also disturbing the acoustic field of ultrasonic waves radiated from the ultrasonic probe. In consideration of these factors, the thickness of the buffer layer 5 may be designed to be substantially ½ a wavelength λ of an ultrasonic wave sent from the transducer unit 1. Incidentally, the thickness of the buffer layer 5 according to the present embodiment is not limited to substantial λ/2. If the geometrical arrangement of transducer elements is stabilized, the buffer layer 5 may be designed to be thicker than substantial λ/2.

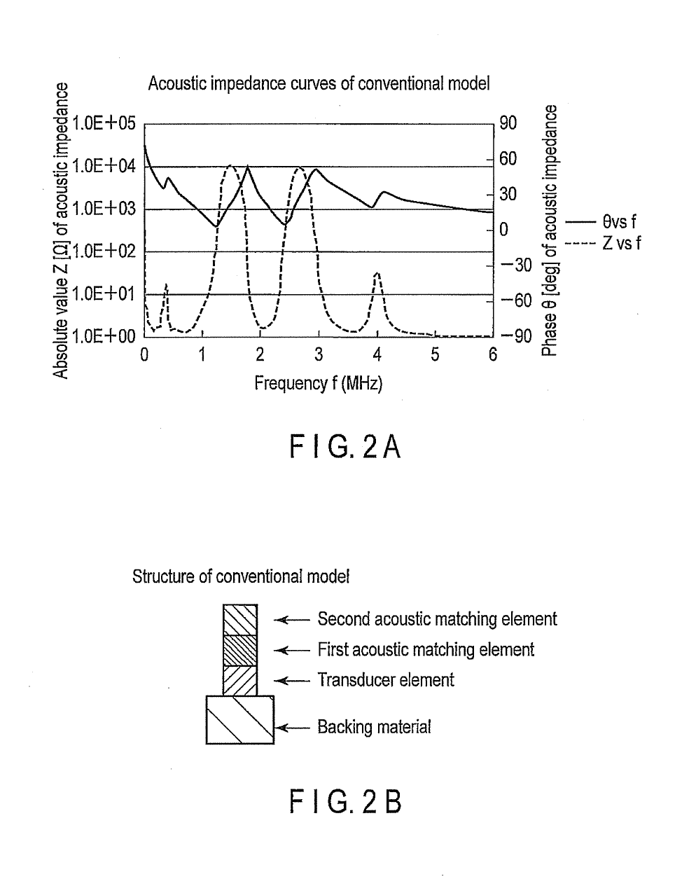

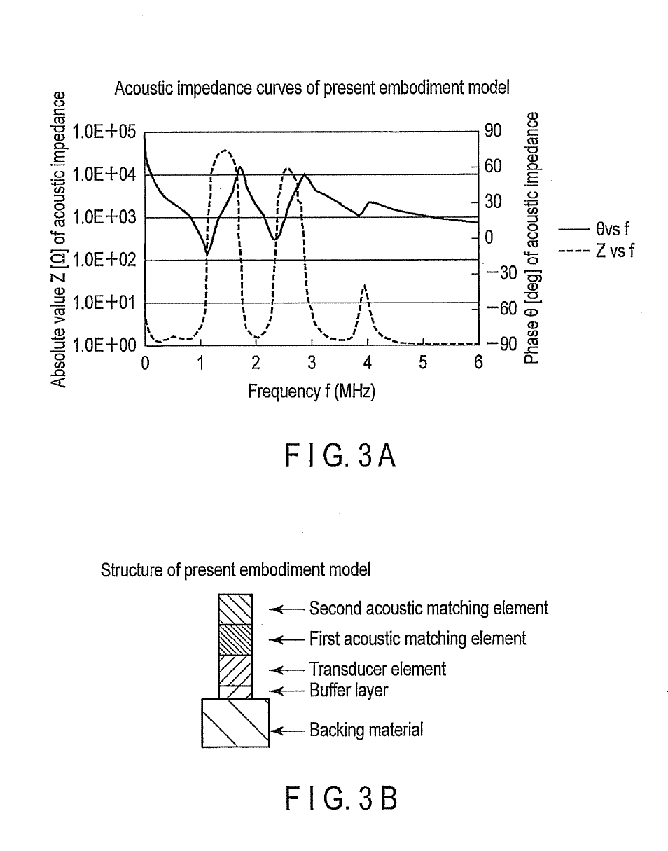

Next, differences of acoustic properties of an ultrasonic probe according to the present embodiment and an ultrasonic probe in a conventional structure will be described with reference to acoustic simulation results by finite-element analysis. FIG. 2 is a diagram showing an acoustic simulation result of an ultrasonic probe in a conventional structure and FIG. 3 is a diagram showing an acoustic simulation result of the ultrasonic probe according to the present embodiment. In an acoustic simulation of the ultrasonic probe in the conventional structure, as shown in FIG. 2B, the impedance of a transducer element is determined by using a laminating structure of the backing material, transducer element, first sound matching element, and second sound matching element as a model. In an acoustic simulation of the ultrasonic probe according to the present embodiment, as shown in FIG. 3B, the impedance of a transducer element is determined by using a laminating structure of the backing material, buffer layer, transducer element, first sound matching element, and second sound matching element as a model. In the acoustic simulations, the acoustic impedance of the buffer layer is equal to the acoustic impedance of the backing material, the thickness of the buffer layer is about λ, and the Poisson ratio of the buffer layer is set to substantial 0.45.

The horizontal axis in FIG. 2A and FIG. 3A is defined as a frequency f [MHz], the left vertical axis is defined as an absolute value Z [Ω] of an acoustic impedance, and the right vertical axis is defined as a phase θ[deg] of an acoustic impedance. A solid line shows a change curve of the phase θ with respect to the frequency f and a dotted line shows a change curve of the absolute value Z with respect to the frequency f. In the change curve in FIG. 2A, three resonance peaks by the transducer element, first acoustic matching layer, and second acoustic matching layer can be identified. A resonance peak is defined as a local maximum value of the absolute value Z or the phase θ. In addition, in the change curve in FIG. 2A, a resonance peak can be identified near 0.4 MHz. The resonance peak near 0.4 MHz results from mechanical vibrations of the backing material, adversely affecting acoustic properties as unnecessary noise. In the change curve in FIG. 3A, on the other hand, three resonance peaks by the piezoelectric transducer element, first acoustic matching layer, and second acoustic matching layer can be identified. In the change curve in FIG. 3A, however, the unnecessary resonance peak near 0.4 MHz disappears. This is because mechanical vibrations of the transducer elements are prevented from being propagated to the backing material by the buffer layer so that mechanical vibrations of the backing material are damped.

With the above configuration, the ultrasonic probe according to the present embodiment has the buffer layer 5 whose Poisson ratio is larger than that of the backing material 6 between the transducer unit 1 and the backing material 6. With such a geometrical arrangement of the transducer unit 1, the buffer layer 5, and the backing material 6, mechanical vibrations from the transducer unit 1 to the backing material 6 can be reduced by the buffer layer 5. In addition, the buffer layer 5 and the backing material 6 are formed so that the acoustic impedances of the buffer layer 5 and the backing material 6 substantially match. In such a case, the frequency of reflection of an ultrasonic wave by the buffer layer 5 can be reduced and the backing material 6 can be caused to absorb unnecessary ultrasonic waves radiated from the transducer unit 1 to the side of the backing material 6. Therefore, the acoustic field is less disturbed by unnecessary ultrasonic waves from the transducer unit 1 so that acoustic properties of the ultrasonic probe can be improved.

Next, the method for manufacturing an ultrasonic probe according to the present embodiment will be described separately as Example 1 and Example 2. In Example 1 and Example 2, the ultrasonic probes have different structures.

### EXAMPLE 1

In Example 1, the buffer layer 5 is arranged between an electrode leading substrate and the backing material 6. The electrode leading substrate is provided to transmit and receive a signal between the main body of ultrasonic diagnostic apparatus and the transducer unit 1 and has a function to lead the front-side electrode and back-side electrode independently for each channel to the outside. For example, a flexible printed wiring board is used as the electrode leading substrate.

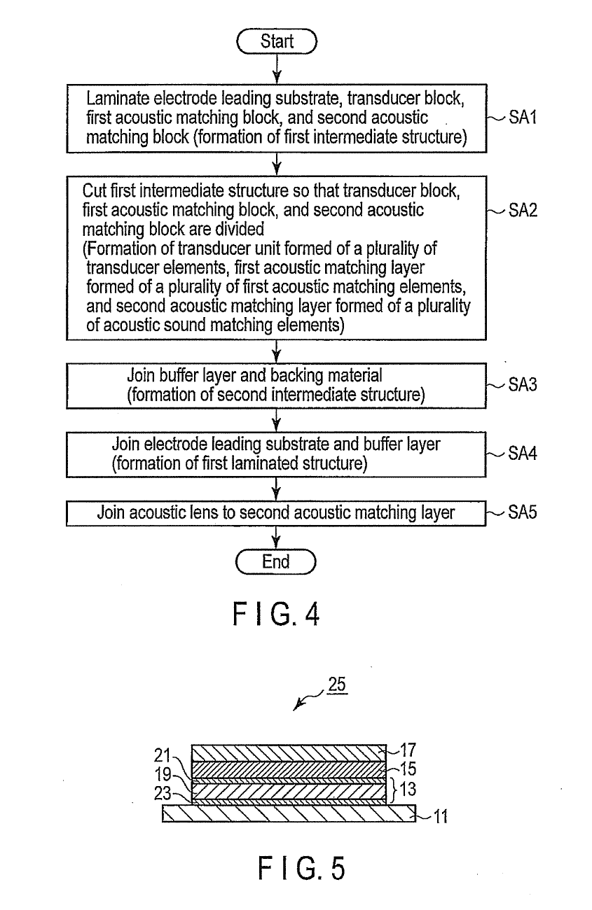

FIG. 4 is a diagram showing the typical flow of a manufacturing process of an ultrasonic probe according to Example 1. In the description that follows, the ultrasonic probe to be manufactured is assumed to be a one-dimensional array type.

First, as shown in FIG. 5, an electrode leading substrate 11, a transducer block 13, a first sound matching block 15, and a second sound matching block 17 are laminated (step SA1). More specifically, the transducer block 13 is joined to the front side of the electrode leading substrate 11, the first sound matching block 15 is joined to the front side of the transducer block 13, and the second sound matching block 17 is joined to the front side of the first sound matching block 15. The transducer block 13 is a structure in which a front-side electrode 21 is formed on the front side of a plate-shaped piezoelectric element 19 and a back-side electrode 23 is formed on the back side thereof. The front-side electrode 21 and the back-side electrode 23 are formed by plating or sputtering of a metal such as gold on both sides of the plate-shaped piezoelectric element 19. The first sound matching block 15 is a plated-shaped structure formed of the material of the first acoustic matching layer 2. The second sound matching block 17 is a plated-shaped structure formed of the material of the second acoustic matching layer 3. An adhesive such as an epoxy adhesive and a silicon adhesive is used to join members. Hereinafter, a laminated body of the electrode leading substrate 11, the transducer block 13, the first sound matching block 15, and the second sound matching block 17 will be called a first intermediate structure. That is, the first intermediate structure will be formed by step SA1.

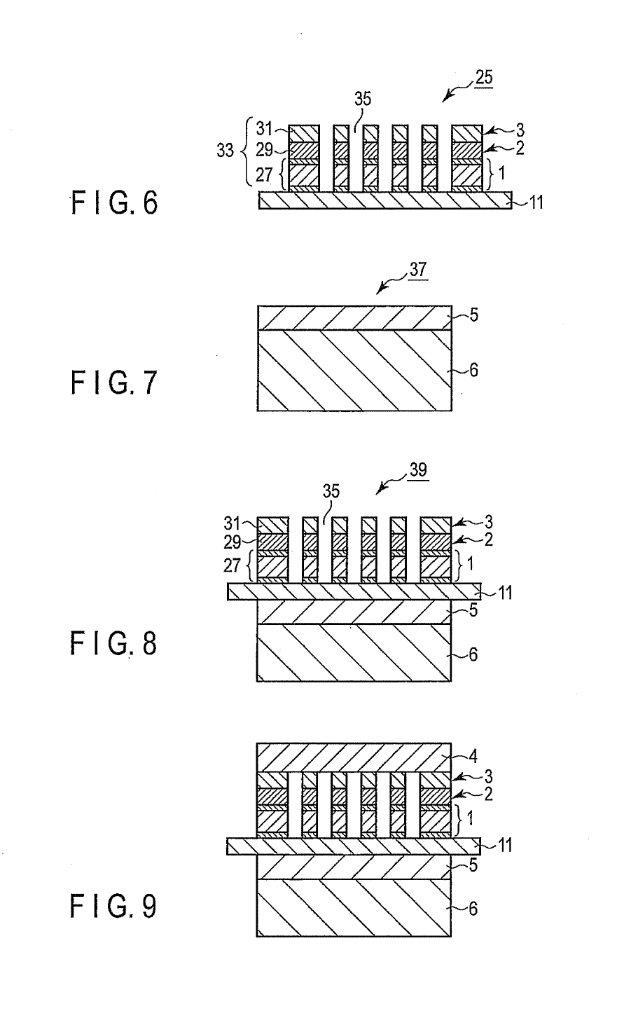

After step SA1 being performed, as shown in FIG. 6, a first intermediate structure 25 is cut with a predetermined cutting pitch along one direction (step SA2). For example, the first intermediate structure 25 is cut from the second acoustic matching layer 3 toward the electrode leading substrate 11. For example, a dicing blade is used for cutting. By the cutting operation, the transducer block 13 is divided into a plurality of transducer elements 27, the first acoustic matching block 15 is divided into a plurality of first acoustic matching elements 29, and the second acoustic matching block 17 is divided into a plurality of second acoustic matching elements 31. As described above, the plurality of transducer elements 27 constitutes the transducer unit 1, the plurality of first acoustic matching elements 29 constitutes the first acoustic matching layer 2, and the plurality of second acoustic matching elements 31 constitutes the second acoustic matching layer 3. A laminated body formed of the transducer element 27, the first acoustic matching element 29, and the second acoustic matching element 31 will be simply called an element 33. A plurality of grooves 35 formed by the cutting operation is arranged with a predetermined cutting pitch. The electrode leading substrate 11 may be completely divided or may not be divided by the cutting operation. However, it is better not to completely divide the electrode leading substrate 11 to prevent a plurality of elements 33 from becoming separated.

On the other hand, as shown in FIG. 7, the backing material 6 and the buffer layer 5 whose Poisson ratio is larger than that of the backing material 6 are joined by, for example, the above adhesive (step SA3). At this point, it is better to set the thickness of the adhesive to, for example, 10 μm or less to reduce the mismatch of acoustic impedance between the buffer layer 5 and the backing material 6 and also to hold the geometrical arrangement of the transducer elements 27 in a good state. Hereinafter, a laminated body of the buffer layer 5 and the backing material 6 will be called a second intermediate structure 37. That is, the second intermediate structure 37 will be formed by step SA3.

After steps SA2 and SA3 being performed, as shown in FIG. 8, the cut first intermediate structure 25 and the second intermediate structure 37 are joined by, for example, the above adhesive in such a way that the electrode leading substrate 11 and the buffer layer 5 are opposed to each other (step SA4). Accordingly, the buffer layer 5 is provided on the back side of the plurality of transducer elements 27 and on the front side of the backing material 6. Hereinafter, a laminated body of the first intermediate structure 25 and the second intermediate structure 37 will be called a first laminated structure 39. That is, the first laminated structure 39 will be formed by step SA4. The groove 35 may be filled with an adhesive such as an epoxy adhesive or silicon adhesive to increase the strength of the transducer unit 1, the first acoustic matching layer 2, and the second acoustic matching layer 3. Metallic particles may be mixed with the adhesive to control thermal expansion of the adhesive with which the groove 35 is filled.

After step SA5 being performed, as shown in FIG. 9, the acoustic lens 4 is joined to the second acoustic matching layer 3 by, for example, the above adhesive (step SA5). Accordingly, the ultrasonic probe is completed.

According to Example 1, the transducer block 13, the first sound matching block 15, and the second sound matching block 17 can be divided into element forms by one cutting operation. Accordingly, compared with Example 2 described later, Example 1 can manufacture an ultrasonic probe by a simple manufacturing process. As described above, the manufacturing process in

FIG. 4 is only an example and the manufacturing process of an ultrasonic probe according to Example 1 is not limited to the above example. For example, joining of the first intermediate structure 25 and the second intermediate structure 37 is not limited to after cutting of the first intermediate structure 25 and may be done before cutting of the first intermediate structure 25. In this case, the laminated body of the first intermediate structure 25 and the second intermediate structure 37 is cut from the side of the second acoustic matching layer toward the backing material 6. From the viewpoint of workability, it is better not to cut the buffer layer 5 and the backing material 6. The reason therefore is, for example, because the buffer layer 5 is soft and the buffer layer 5 may not be cut with precision. Another reason is that the buffer layer 5 is bent during cutting, reducing adhesive strength between the transducer element 27 and the buffer layer 5. Incidentally, if the cutting precision of the buffer layer 5 is good, the buffer layer 5 and the backing material 6 may be cut.

In the above manufacturing process, the buffer layer 5 is assumed to be joined to the backing material 6 in advance before being joined to the first intermediate structure 25. However, before the buffer layer 5 and the backing material 6 being joined, the buffer layer 5 may be joined to the back side of the electrode leading substrate 11. In this case, even if the electrode leading substrate 11 is cut, the plurality of elements 33 will not become separated by the cutting if the buffer layer 5 is not cut. Therefore, the manufacture of an ultrasonic probe is made easier.

In the above manufacturing process, the ultrasonic probe is assumed to be a one-dimensional array type. However, the ultrasonic probe according to Example 1 may be a two-dimensional array type. In this case, the first intermediate structure 25 is cut, for example, in step SA2 in a lattice shape with a predetermined cutting pitch. By cutting the first intermediate structure 25 two-dimensionally, the transducer block 13, the first sound matching block 15, and the second sound matching block 17 are divided two-dimensionally. Accordingly, ultrasonic probes of two-dimensional array type can be manufactured.

### EXAMPLE 2

In Example 1, as described above, the buffer layer 5 is not directly in contact with the transducer element 27. Thus, an ultrasonic wave traveling from the transducer element 27 toward the backing material 6 may be reflected by a boundary surface between the electrode leading substrate 11 and the buffer layer 5. This could lead to degradation of the acoustic field of ultrasonic waves. In Example 2, an electrode leading substrate 11 is not arranged between a transducer unit 1 and a buffer layer 5 and is provided instead on the side surface of a transducer unit 1.

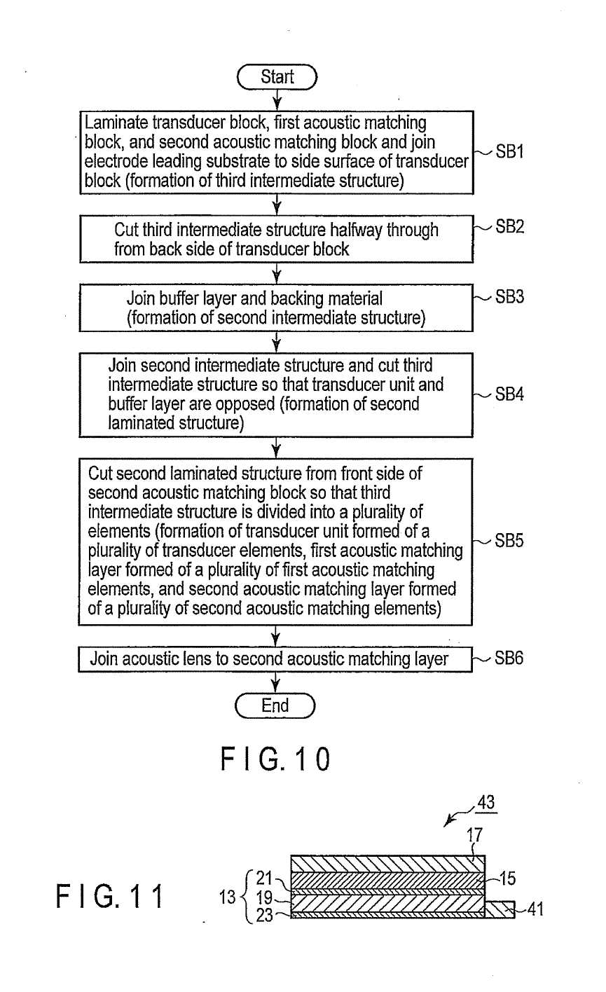

FIG. 10 is a diagram showing the typical flow of the manufacturing process of an ultrasonic probe according to Example 2. In the description that follows, the ultrasonic probe to be manufactured is assumed to be a one-dimensional array type.

First, as shown in FIG. 11, a transducer block 13, a first acoustic matching block 15, and a second acoustic matching block 17 are laminated and an electrode leading substrate 41 is joined to the side surface of the transducer block 13 (step SB1). More specifically, the first acoustic matching block 15 is joined to the front side of the transducer block 13 and the second acoustic matching block 17 is joined to the front side of the first acoustic matching block 15. Then, the electrode leading substrate 41 is joined to the side surface of the transducer block 13. Like in Example 1, for example, an adhesive such as an epoxy adhesive and a silicon adhesive is used to join each member. Hereinafter, a laminated body of the electrode leading substrate 41, the transducer block 13, the first acoustic matching block 15, and the second acoustic matching block 17 will be called a third intermediate structure 43. That is, the third intermediate structure 43 will be formed by step SB1.

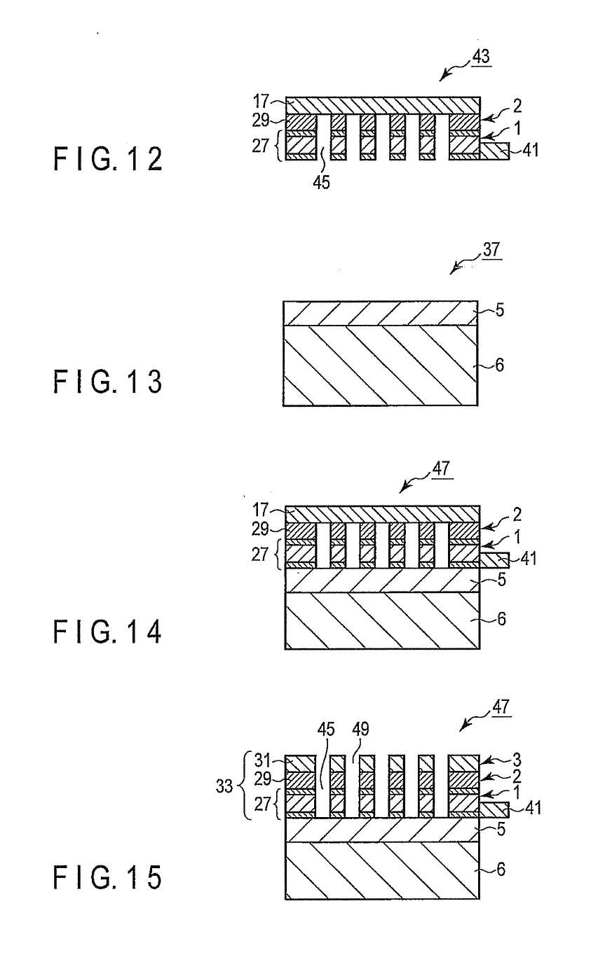

After step SB1 being performed, as shown in FIG. 12, the third intermediate structure 43 is cut with a predetermined cutting pitch in one direction from the back side of the transducer block 13 up to halfway through the third intermediate structure 43 (step SB2). In step SB2, the third intermediate structure 43 is cut so as not to be completely divided into element forms. For example, in FIG. 12, the third intermediate structure 43 is cut up to the boundary between the first acoustic matching block 15 and the second acoustic matching block 17. In this case, a plurality of transducer elements 27 is formed from the transducer block 13 and a plurality of first acoustic matching elements 29 is formed from the first acoustic matching block 15. As described above, the plurality of transducer elements 27 constitutes the transducer unit 1 and the plurality of first acoustic matching elements 29 constitutes a first acoustic matching layer 2. However, the depth of a groove 45 according to the present embodiment is not limited to the above example. The groove 45 may be formed up to any depth if the third intermediate structure 43 is not completely separated by cutting. For example, though not shown, the third intermediate structure 43 may be cut up to the boundary between the transducer block 13 and the first acoustic matching block 15, halfway through the transducer block 13, halfway through the first acoustic matching block 15, or halfway through the second acoustic matching block 17. Like Example 1, a dicing blade is used for cutting.

On the other hand, as shown in FIG. 13, a backing material 6 and the buffer layer 5 whose Poisson ratio is larger than that of the backing material 6 are joined by, for example, the above adhesive (step SB3). A second intermediate structure 37 will be formed by step SB3. Step SB3 is the same as step SA3 and so the description thereof is omitted.

After steps SB2 and SB3 being performed, as shown in FIG. 14, the second intermediate structure 37 and the cut third intermediate structure 43 are joined in such a way that the buffer layer 5 is in contact with the transducer unit 1 (the transducer block 13 if the transducer block 13 is not completely cut in step SB2) (step SB4). Hereinafter, a laminated body of the third intermediate structure 43 and the second intermediate structure 37 will be called a second laminated structure 47.

After steps SB4 being performed, as shown in FIG. 15, the third intermediate structure 43 is cut from the front side of the second acoustic matching block 17 so that the third intermediate structure 43 is completely divided into a plurality of elements 33 (step SB5). More specifically, the cutting portion in step SB5 is positioned so that a groove 49 newly formed in step SB 5 and the groove 45 formed in step SB2 communicate. Then, the positioned cutting portion is cut by a dicing blade or the like. In other words, the third intermediate structure 43 is cut from the front side of the second acoustic matching block 17 up to the groove 45 until the groove 49 is connected to the groove 45. Accordingly, the transducer block 13, the first acoustic matching block 15, and the second acoustic matching block 17 are divided into the plurality of elements 33. In FIG. 14, in step SB5, the second acoustic matching block 17 is cut to form a plurality of second acoustic matching elements 31 from the second acoustic matching block 17. As described above, the plurality of second acoustic matching elements 31 constitutes a second acoustic matching layer 3. The grooves 45, 49 may be filled with an adhesive such as an epoxy adhesive or silicon adhesive to increase the strength of the transducer unit 1, the first acoustic matching layer 2, and the second acoustic matching layer 3. Metallic particles may be mixed with the adhesive to control thermal expansion of the adhesive with which the grooves 45, 49 is filled.

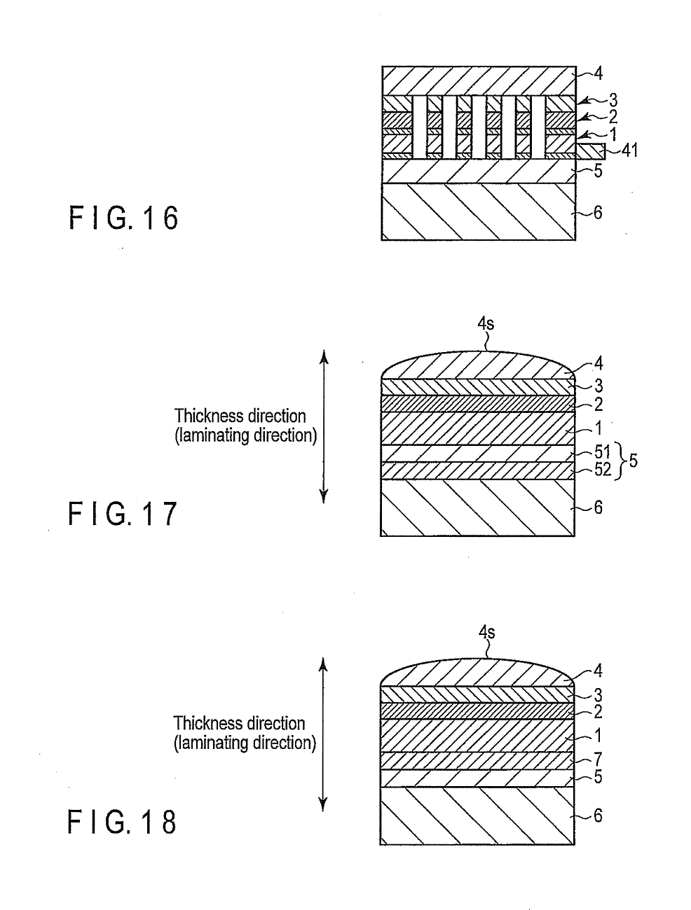

After step SB5 being performed, as shown in FIG. 16, an acoustic lens 4 is joined to the second acoustic matching layer 3 by the above adhesive (step SB6). Accordingly, the ultrasonic probe is completed.

According to Example 2, the cutting process of dividing the third intermediate structure 43 formed of the transducer block 13, the first acoustic matching block 15, and the second acoustic matching block 17 into element forms divides into two steps. The reason therefore is as follows.

An ultrasonic probe according to Example 2 has a structure in which the buffer layer 5 is directly in contact with the transducer unit 1. If the third intermediate structure 43 should be divided into element forms in one cutting operation, for example, the third intermediate structure 43 should be divided into the plurality of elements 33 in step SB2, the transducer element 27, the first acoustic matching element 29, and the second acoustic matching element 31 become separated due to the lack of foundation. To prevent the transducer element 27, the first acoustic matching element 29, and the second acoustic matching element 31 from becoming separated, in step SB5, the buffer layer 5 is used as a foundation to cut the third intermediate structure 43. However, the buffer layer 5 has a large Poisson ratio and is flexible. Thus, if the third intermediate structure 43 is cut by using the buffer layer 5 as a foundation, the third intermediate structure 43 may become geometrically unstable with respect to the buffer layer 5, which makes successful element division impossible.

According to Example 2, as shown in steps SB2 and SB5, the cutting process is divided into two steps. That is, in step SB2, the third intermediate structure 43 is cut halfway through, in step SB4, the buffer layer 5 and the cut third intermediate structure 43 are joined, and in step SB5, the remaining cutting portion of the third intermediate structure 43 is cut. By dividing the cutting process into two steps as described above, the third intermediate structure 43 can be divided into elements with precision without causing the transducer element 27, the first acoustic matching element 29, and the second acoustic matching element 31 to become separated.

In the above manufacturing process, the ultrasonic probe is assumed to be a one-dimensional array type. However, the ultrasonic probe according to Example 2 may be a two-dimensional array type. In this case, the third intermediate structure 43 is cut, for example, in step SB2, halfway through in a lattice shape with a predetermined cutting pitch and, in step SB5, the remaining cutting portion is cut. By cutting the third intermediate structure 43 two-dimensionally, the transducer block 13, the first acoustic matching block 15, and the second acoustic matching block 17 are divided two-dimensionally. Accordingly, ultrasonic probes of two-dimensional array type can be manufactured.

### (Modification 1)

In the above description, the buffer layer 5 is assumed to have a one-layer structure. However, the buffer layer 5 according to the present embodiment is not limited to the above example. The buffer layer 5 may have a structure of two layers or more. An ultrasonic probe according to Modification 1 will be described below. In the description that follows, the same reference numerals are attached to structural elements having substantially the same function as in the present embodiment and a duplicate description will be provided only if necessary. A buffer layer 5 according to Modification 1 is assumed to have, as an example, a two-layer structure.

FIG. 17 is a diagram showing the schematic structure of an ultrasonic probe according to Modification 1. As shown in FIG. 17, the buffer layer 5 has a two-layer structure formed of a first layer 51 and a second layer 52. The first layer 51 is provided in a position closer to the transducer unit 1 than the second layer 52. At least one of the first layer 51 and the second layer 52 has a Poisson ratio larger than that of the backing material to damp mechanical vibrations of the transducer unit 1. To enhance damping efficiency of mechanical vibrations of the transducer unit 1, the buffer layer 5 may be provided in such a way that the layer (first layer 51) closer to the transducer unit 1 has a Poisson ratio larger than that of the layer (second layer 52) farther from the transducer unit 1.

If the buffer layer 5 has a structure of three layers or more, the buffer layer 5 may similarly be provided in such a way that the layer closest to the transducer unit 1 has a Poisson ratio larger than that of any other layer.

Thus, according to Modification 1, degradation of acoustic properties involved in vibration of a transducer element can be prevented.

### (Modification 2)

In the above description, if the buffer layer 5 is provided on the back side of the transducer unit 1, another structural element may be provided or may not be provided between the transducer unit 1 and the buffer layer 5. In the above description, for example, the FPC 11 is cited as a structural element provided between the transducer unit 1 and the buffer layer 5. However, in addition to the FPC 11, any structural element may be provided between the transducer unit 1 and the buffer layer 5 in an ultrasonic probe according to the present embodiment. An ultrasonic probe according to Modification 2 will be described below. In the description that follows, the same reference numerals are attached to structural elements having substantially the same function as in the present embodiment and a duplicate description will be provided only if necessary.

FIG. 18 is a diagram showing the schematic structure of an ultrasonic probe according to Modification 2. As shown in FIG. 18, a third acoustic matching layer 7 is provided between a transducer unit 1 and a buffer layer 5. The third acoustic matching layer 7 is provided to reduce an acoustic impedance mismatch between the transducer unit 1 and the buffer layer 5. An ultrasonic wave may be radiated to the back side of the transducer unit 1. An ultrasonic wave radiated to the back side of the transducer unit 1 can be made to be efficiently transmitted to the buffer layer 5 and a backing material 6 via the third acoustic matching layer 7.

Incidentally, structural elements provided between the transducer unit 1 and the buffer layer 5 are not limited to the FPC 11 and the third acoustic matching layer 7 and structural elements having any function, structure, and composition may be provided.

Thus, according to Modification 2, degradation of acoustic properties involved in vibration of a transducer element can be prevented.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic probe comprising:
a transducer element that vibrates to transmit and receive an ultrasonic wave;
a buffer layer provided on a back side of the transducer element; and
a backing material provided on a back side of the buffer layer to damp the ultrasonic wave from the transducer element,
wherein the buffer layer has a Poisson ratio larger than the Poisson ratio of the backing material.

2. The ultrasonic probe according to claim 1, wherein the Poisson ratio of the buffer layer is 0.4 or more.

3. The ultrasonic probe according to claim 1, wherein a difference between an acoustic impedance of the buffer layer and the acoustic impedance of the backing material is −20% or more and +20% or less.

4. The ultrasonic probe according to claim 1, wherein the buffer layer has a thickness substantially half a wavelength of the ultrasonic wave transmitted from the transducer element or more.

5. The ultrasonic probe according to claim 1, wherein the buffer layer has two layers or more.

6. The ultrasonic probe according to claim 5, wherein at least one layer of the buffer layer comprising the two layers or more has the Poisson ratio larger than the Poisson ratio of the backing material.

7. The ultrasonic probe according to claim 6, wherein the layer closer to the transducer element of the buffer layer of the two layers or more has the Poisson ratio larger than the Poisson ratio of the layer farther from the transducer element of the buffer layer of the two layers or more.

8. An ultrasonic probe manufacturing method, comprising:
forming a structure comprising a plate-shaped transducer block and an acoustic matching block;
cutting the structure to divide the structure into a plurality of elements; and
joining the cut structure and a buffer layer having flexibility so that the buffer layer is provided on a back side of the transducer block.

9. An ultrasonic probe manufacturing method, comprising:
forming a structure comprising a plate-shaped transducer block and an acoustic matching block;
cutting the structure from the back side of the transducer block to divide the structure into a plurality of elements;
joining the cut structure and a buffer layer having flexibility so that the buffer layer comes into contact with a back side of the transducer block; and
cutting the joined structure and buffer layer from a front side of the acoustic matching block to divide the structure into a plurality of elements in such a way that a groove formed in the structure by the cutting and a newly formed groove communicate.
